(19) United States
(12) Patent Application Publication
(10) Pub. No.: US 2026/0297660 A1
(43) Pub. Date: Oct. 1, 2026

(54) PRE-GRAFTED HYDROGEL FOR IMPROVED SOLID SUPPORT SURFACE ADHESION

(71) Applicant: Illumina, Inc., San Diego, CA (US)

(72) Inventors: Daniel Wright, San Diego, CA (US); Mike Rapp, San Diego, CA (US); Graham Boud, San Diego, CA (US); Avishek Aiyar, San Diego, CA (US)

(73) Assignee: Illumina, Inc., San Diego, CA (US)

(21) Appl. No.: 19/575,065

(22) Filed: Mar. 23, 2026

Related U.S. Application Data
(60) Provisional application No. 63/778,074, filed on Mar. 26, 2025.

(51) Int. Cl. C12Q 1/6834; C12Q 1/6825; G01N 21/64

(52) U.S. Cl. CPC C12Q 1/6834; C12Q 1/6825; G01N 21/6428; G01N 2021/6439

(57) ABSTRACT

Functionalized solid support surfaces are disclosed for the detection of a polynucleotide sample, in which the surface includes a polymeric hydrogel grafted with an adhesion promoting biopolymer configured to covalently bind a capture primer. Also disclosed are biological assays and methods of detection of a polynucleotide sample utilizing functional solid support surfaces herein disclosed.

### CROSS REFERENCE TO RELATED APPLICATIONS

This application claims priority to U.S. provisional patent application No. 63/778,074, which was filed Mar. 26, 2025, which is incorporated herein by reference in its entirety.

### SEQUENCE LISTING

The instant application contains a Sequence Listing which has been submitted herewith and is hereby incorporated by reference in its entirety. Said .xml copy, created on Mar. 18, 2026, is named I03110246, and is 4,802 bytes in size.

### BACKGROUND

Flow cells and other solid support systems are used in a variety of biological assays, including assays for sequencing of a biopolymeric sample in connection with various genomic, epigenomic, and transcriptomic analyses, including high-throughput sequencing implemented using Next Generation Sequencing (NGS). Sequencing methodologies for polynucleotide materials on NGS platforms commonly deploy deoxyribonucleic acid (DNA) libraries in which a DNA target (e.g., genomic DNA (gDNA), or complimentary DNA (cDNA)) is processed into fragments and ligated with technology-specific adaptors. NGS workflow using, e.g., a sequence-by-synthesis (SBS) technique, involves loading a DNA library onto a flow cell and hybridizing individual DNA fragments to adapter-specific complimentary oligonucleotides (oligo primers) covalently bound to the functionalized area(s) of the flow cell surface (planar or patterned); clustering the individual fragments into thousands of identical DNA template strands (amplicons) through amplification (e.g., bridge or exclusion); and, finally, sequencing, in which copy strands are simultaneously synthesized and sequenced on the DNA templates using a reversible terminator-based process that detects signals emitted from fluorophore-labeled single bases as they are added round-by-round to the copy strands. Because the multiple template strands of each cluster have the same sequence, base pairs incorporated into the corresponding copy strands in each round will be the same, and thus the signal generated from each round will be enhanced proportional to the number of copies of the template strand in the cluster.

For polynucleotide sequencing as well as other type nucleic acid analyses, robust surface adhesion of oligo primers and other such capture agents may be desirable to maintain the functionalized area(s) of the solid support throughout various cycles of analysis and/or throughout the lifetime of the solid support system before and during a variety of uses. To that end, there is a need to improve surface adhesion so that the solid support system can improve output by (a) minimizing data quality defects (i.e. padhopping/duplicates or data loss due to poor surface adhesion) and/or (b) improving the extensibility for long reads/cycles by increasing surface attachment robustness.

### SUMMARY

The present disclosure relates to functionalized solid supports for analyzing a nucleic acid sample. In one aspect, a functionalized solid support surface is provided, wherein the solid support surface comprises a hydrogel layer adhered to the surface and the hydrogel contains one or more adhesion promoting biopolymers capable of binding a capture primer, wherein a nucleic acid sample may be immobilized on the functional solid support surface through interaction with the capture primer.

According to certain embodiments, the adhesion promoting biopolymer can be oligonucleotide or mixture of oligonucleotides. By way of example, each such oligonucleotide or respective oligonucleotide in a mixture can include one of following nucleobase sequences:

(SEQ ID No: 1)
TTTTTTTTTTAATGATACGGCGACCACCGAGAUCTACAC,
(SEQ ID No: 2)
TTTTTTAATGATACGGCGACCACCGAGA/vinyl-T/CTACAC,
and
(SEQ ID NO: 3)
TTTTTTTTTTCAAGCAGAAGACGGCATACGAGAT.

The adhesion promoting biopolymer consistent with the disclosure can also be peptide. By way of example, peptides herein may include post-translationally modified amino acids or sequence mixtures of amino acids. According to certain examples the peptide is a biomimetic peptide. Such biomimetic peptides can include (1) polydopamine, (2) domains of poly-tyrosine with post-translational modification from tyrosine to 3,4-dihydroxyphenylalanine (also known as L-DOPA, and abbreviated as “Y*”), and/or (3) repeat units of Y*-X, where X is a cationic amino acid such as lysine, arginine, or histidine. 4.

In certain embodiments, the hydrogel herein can be synthesized from natural polymers, synthetic polymers, polymerizable synthetic monomers, or a combination of natural and synthetic polymers. The hydrogel according to examples can be a poly(acrylamide), poly(methacrylate), poly(acrylate), poly(methacrylamide), polyethylene glycol, and/or polypropylene glycol.

The solid support surface according to examples can be composed of any low-background, reagent resistant material suitable for use in analyzing a nucleic acid sample in a fluorescent-, chemiluminescent-, colorimetric-, electrochemical-, or electrooptic-based assays, including epoxy silane, glass, silicone or silicone polymers (poly-dimethylsiloxane (PDMS)), plastics (e.g., acrylics, polystyrene, cyclic olefins), polyamides, polycarbonates, polyimides, silica, and the like, or any appropriate high-index, dielectric materials for use in photonic or plasmonic-based assays, including, e.g, metal oxides, nitrides or phosphides, transition metals, ceramics, and the like.

In some embodiments, the functionalized solid support surface herein can be supported on a microfluidic architecture such as flow cells, microarrays, and integrated microfluidic circuits or imaging-generating chips for use, e.g., with solid state imaging systems, including complementary metal oxide semiconductor (CMOS) and charge-coupled device (CCD) sensor systems.

A flow cell architecture for supporting the functionalized solid support surface consistent with the disclosure can be patterned or unpatterned. In certain examples, a patterned flow cell can be utilized in which the functionalized solid support surface is coterminous with an array of discrete reaction sites separated by interstitial regions of featureless flow cell substrate. In examples, each discrete rection site can be formed in a nanowell or other depression in the substrate of the flow cell. In one example, the flow cell substrate includes an optic support layer for performing optical detection a nucleic acid sample.

The present disclosure further relates to biological assays performed on functionalized solid support surfaces described herein. In certain embodiments, the functionalized solid support surface can be incorporated in a biological assay useful for the detection of a nucleic acid sample or constituent analyte(s) thereof, in which the functionalized solid support surface is seeded with an oligonucleotide capture primer configured to hybridize to a target nucleic acid material or constituent analyte, and wherein each primer is attached to a respective adhesion promoting biopolymer of the hydrogel. By way of example, the biological assay may include (1) a functionalized solid support surface, wherein the solid support surface comprises a hydrogel layer adhered to the surface, wherein the hydrogel contains one or more adhesion promoting biopolymers; (2) a capture primer bound to the solid support surface through interaction with the one or one or more adhesion promoting biopolymers; and (3) a nucleic acid sample immobilized on the solid support surface via interaction with the capture primer.

In certain examples, the adhesion promoting biopolymer can be an oligonucleotide or oligonucleotide mixture. For example, each such oligonucleotide or respective oligonucleotide in a mixture can include one of following nucleobase sequences:

(SEQ ID No: 1)
TTTTTTTTTTAATGATACGGCGACCACCGAGAUCTACAC,
(SEQ ID No: 2)
TTTTTTAATGATACGGCGACCACCGAGA/vinyl-T/CTACAC,
and
(SEQ ID NO: 3)
TTTTTTTTTTCAAGCAGAAGACGGCATACGAGAT.

In the same or other examples, the adhesion promoting biopolymer consistent with the disclosure can also be an amino acid. For example, peptides herein may include post-translationally modified amino acids, sequence mixtures of amino acids, and/or post-translationally modified amino acids. According to certain examples the peptide is a biomimetic peptide. Such biomimetic peptides can include (1) polydopamine, (2) domains of poly-tyrosine with post-translational modification from tyrosine to 3,4-dihydroxyphenylalanine (also known as L-DOPA, and abbreviated as “Y*”), and/or (3) repeat units of Y*-X, where X is a cationic amino acid such as lysine, arginine, or histidine. In yet other examples, the adhesion promoting biopolymer consists of a mixture of adhesion peptides and oligos.

In one example, a biological assay implemented on a functionalized solid support surface herein can be utilized in a method for optical detection of a nucleic acid material or one or more of constituent analytes. The method can include providing an optical detection system comprising an excitation source, one or more optical sensors, and a signal processor. In example embodiments, the method can further include irradiating, via the excitation source, at least a portion of the functionalizes support surface of the biological assay with an incident light; detecting, via the one or more optical sensors, an output signal emitted by the nucleic acid sample or constituent analyte(s) as an optic field response to the incident light, and obtaining from the output signal, via the signal processor, data indicative of a characteristic of the sample or constituent analyte(s).

In one example, a method for optical detection of a nucleic acid sample in a sequencing protocol is provided. Here, flow cells provide a convenient format in the example sequencing protocol, which can involve multiple cycles of repeated chemical delivery and image capture. According to the example method, a DNA sample (e.g., gDNA) or an RNA sample (e.g., as cDNA) in the form of a DNA library can be obtained in which the DNA sample is processed into constituent fragment strands and ligated with technology-specific adaptors. According to the example method, the DNA library can be loaded onto a patterned functionalized solid support surface supported on a flow cell device, where constituent fragment strands are flowed across an array of nanowells impressed in an optic support layer of the flow cell substrate and individual strands are absorbed by nanowells of the array at a substantially 1:1 basis, where each absorbed strand is immobilized at a reaction site of respective nanowells through interaction between the strand adaptor and a capture primer bound to the adhesion promoting biopolymer grafted to the hydrogel coating each reaction site. Each immobilized strand can then be amplified (e.g., using bridge or exclusion amplification) to yield a substantially monoclonal cluster of template strands within each respective nanoantennae.

Each of the resulting clusters is then sequenced using, e.g., a sequencing-by-synthesis (SBS) technique. SBS in general involves the enzymatic extension of a nascent copy strand through iterative addition and simultaneous detection of nucleoside monomers against a template strands. For each iteration (or cycle), labeled nucleoside monomers can be detected through induced fluorescence as each monomer is added to the copy strand then replaced in a reverse-terminator process with non-labeled analogues before the start of a subsequent cycle. According to the example method, in a first cycle, (1) a buffer solution containing a mixture of four different labeled nucleoside derivatives (one for each of the four DNA nucleobases (A, G, C, T)) can be provided to a cluster of template strands in the presence of polymerase; (2) a complementary nucleoside derivative can be added to a nascent copy strand hybridized to each template strand via polymerase primer extension; (3) the added nucleoside derivatives can be irradiated with incident light via an excitation source to induce fluorescence and emission of an output signal from the cluster; (4) the output signal can be detected by one or more optical sensors through imaging of the signal as a point source at an addressed location on the optic substrate; and (5) information imparted in the imaged output signal can be processed by a signal processor to record the nucleobase of the added nucleoside derivative and an address of the cluster on the optic substrate. Steps (1)-(5) can be performed simultaneously for a plurality of clusters with a given frame (or tile) of the one or more optic sensors. Steps (1)-(5) can then be repeated in subsequent cycles to n number of total cycles, where n is equal to the size of the template strands in base pairs (bp).

### BRIEF DESCRIPTION OF THE DRAWINGS

FIG. 1 presents a schematic of pre-grafted hydrogel structure for improved adhesion.

FIG. 2 presents a schematic of pre-grafted hydrogels for improved adhesion within flow cell nanowells vs current spin coat and graft deposition. Note the direct interaction of the adhesion primers with the flow cell surface for the pre-grafted hydrogel.

FIG. 3 shows a schematic of pre-grafted hydrogels flow cell fabrication workflows.

FIG. 4A-B is a top view of example flow cell systems.

FIG. 5 shows an illustration of workflow for hydrogel-hydrogel bonded glass slides.

FIG. 6 presents an example of debond shear force plots of pre-grafted poly acrylamide and ungrafted polyacrylamide (top) and debond force vs sample type for both dry and water soaked debond tests (bottom).

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein. Those of ordinary skill in the art will recognize a variety of equivalent variations on the description that follows.

The following detailed description is made with reference to the figures. Example implementations are described to illustrate the technology disclosed, not to limit its scope, which is defined by the claims. In that regard, disclosure of various exemplary sequencing implementations for use in connection with sequencing-by-synthesis (SBS) for Next Generation Sequencing (NGS) is meant to illustrate the implementations in the context of a well-known and widely-used sequencing and imaging techniques. The functionalized solid support technology described herein may be used in conjunction with a variety of other nucleic acid sequencing techniques. Particularly applicable techniques are those in which nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are also applicable. In this particular context, various implementations herein have application to any number of other sequencing techniques, including, e.g., real time sequencing; nanopore sequencing; long read sequencing; single-molecule sequencing; stochastic sequencing; amplification-free sequencing; sequencing by ligation; pyrosequencing; and ion semiconductor sequencing.

In the context of functionalized solid phase supports, generally, and the adhesion promoting biopolymers, in particular, one of ordinary skill in the art will recognize consistent usage in the context of polynucleotide detection and analysis beyond the milieu of SBS and other sequencing techniques. For example, functionalized solid phase supports coated with hydrogel containing adhesion promoting biopolymers herein may utilized in nucleic acid expression and/or detection methodologies for the detection of single nucleotide polymorphs (SNIPS) and/or insertions/deletions (Indels) in a sample genome, e.g., genotyping. Other appropriate methodologies include fluorescence resonance energy transfer (FRET), time-resolved FRET (TR-FRET), bimolecular fluorescence complementation (BiFC), green fluorescent protein GFP fluorescence, fluorescence activated cell sorting (FACS) and fluorescence intensity (FLINT)/Fluorescence Intensity Ratio (FIR), to name a few.

In the context of fluorescence-based detection, various types of fluorescence microscopy may be used with system embodiments described herein. Fluorescence microscopy may be performed using an optical detection system that includes a light source (e.g., lasers, light emitting diodes (LEDs)) tuned to wavelengths of light that induce excitation in the fluorescent dyes used for labelling a sample biological material or probe; one or more optical instruments, such as cameras, lenses, sensors, to capture signals emitted through induced excitation, and one or more processors for developing composite images from captured signals emitted from labelled targets within the optical elements' field of view (tile) in a given sequencing assay. For example, embodiments may be configured to perform at least one of conventional fluorescent imaging, epifluorescence imaging, total-internal-reflectance-fluorescence (TIRF) imaging, a time-delay integration (TDI) imaging (CCD-TDI or CMOS-TDI), or Super Resolution imaging, e.g., Structured Illumination Microscopy (SIM). Furthermore, the imaging sessions may include line scanning one or more samples such that a linear focal region of light is scanned across the sample(s). Imaging sessions may also include moving a point focal region of light in a raster pattern across the sample(s). Alternatively, one or more regions of the sample(s) may be illuminated at one time in a step and shoot manner.

Moreover, while fluorescence based detection methodologies are presented to illustrate the utility of the functionalized solid phase support systems of the disclosure, other detection methodologies for solid phase detection and/or analysis of nucleic acid materials or their constituents may also be deployed consistent with the disclosure including chemiluminescent-, colorimetric-, electrochemical-, or electrooptic-based methodologies. Similarly, while flow cell architecture is used as a convenient format for supporting the functionalized solid phase surfaces herein, other microfluidic architecture or other support structure may be employed, including, e.g., microarrays, bead arrays, microwells, microplates, and integrated microfluidic circuits for use, e.g., with solid state imaging systems, including complementary metal oxide semiconductor (CMOS) and charge-coupled device (CCD) sensor systems.

In certain embodiments, optical detection systems may process signals into spectral data through spectroscopy, using various techniques known in the art, including, e.g., Raman spectroscopy, including surface-enhanced Raman spectroscopy (SERS), and up-conversion spectroscopy. SERS is a spectroscopic technique that can enhance the otherwise feeble Raman scattering effect with the help of plasmonic or dielectric nanoantennae. The enhancement effect originates due to an increase in the local electric field magnitude. SERS allows for the structural fingerprinting of low-concentration analytes through the plasmon-mediated amplification of electrical fields.

Nucleic acid materials may be referred to herein as “nucleic acids,” “nucleic acid molecules,” “nucleic acid materials,” “nucleic acid sequences,” “polynucleotides,” or “oligonucleotides,” and can comprise a polymeric form of nucleotides of any length, can comprise DNA and/or RNA, and can be single-stranded, double-stranded, or multiple stranded. One strand of a nucleic acid also refers to its complement. Nucleic acid analytes may be gDNA, including DNA variants (e.g., alleles, polymorphs, missense), mtDNA, mRNA, cDNA transcribed from mRNA, non-coding RNA, and small RNA. Nucleic acid materials herein may also include polynucleotide analogues, amplicons, conjugates, and substitutions, crosslinked polynucleotides, polynucleotide complexes, and non-natural polynucleotides, including, but not limited to, dideoxynucleotides, or biotinylated, aminated, deaminated, alkylated, benzylated, flourophor-labeled polynucleotides.

Nucleic acids in certain implementations may include, for instance, linear polymers of deoxyribonucleotides in 3′-5′ phosphodiester or other linkages, such as DNA, for example, single- and double-stranded DNA, genomic DNA, copy DNA or complementary DNA (cDNA), recombinant DNA, or any form of synthetic or modified DNA. In other implementations, nucleic acids include for instance, linear polymers of ribonucleotides in 3′-5′ phosphodiester or other linkages such as ribonucleic acids (RNA), for example, single- and double-stranded RNA, messenger (mRNA), copy RNA or complementary RNA (cRNA), alternatively spliced mRNA, ribosomal RNA, small nucleolar RNA (snoRNA), microRNAs (miRNA), small interfering RNAs (sRNA), piwi RNAs (piRNA), or any form of synthetic or modified RNA. Nucleic acids used in the compositions and methods of the present invention may vary in length and may be intact or full-length molecules or fragments or smaller parts of larger nucleic acid molecules. In particular implementations, a nucleic acid may have one or more detectable labels, as described elsewhere herein.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as the peptide-oligo conjugates as described herein.

In various implementations, nucleic acids may be used as templates as provided herein (e.g., a nucleic acid template, or a nucleic acid complement that is complementary to a nucleic acid nucleic acid template) for particular types of nucleic acid analysis, including but not limited to nucleic acid amplification, nucleic acid expression analysis, and/or nucleic acid sequence determination or suitable combinations thereof.

In some implementations, the nucleic acid may comprise a plurality of copies of template nucleic acid and/or complements thereof, attached via their 5′ termini to the solid support. Such nucleic acid materials may be referred to “clusters” “colonies,” or “clonal populations.” The copies of nucleic acid strands making up the nucleic acid clusters may be in a single or double stranded form. Copies of a nucleic acid template that are present in a cluster can have nucleotides at corresponding positions that differ from each other, for example, due to presence of a label moiety. The corresponding positions can also contain analog structures having different chemical structure but similar Watson-Crick base-pairing properties, such as is the case for uracil and thymine. Nucleic acid clusters can optionally be created on solid supports by amplification, including, e.g., bridge amplification or exclusion amplification (ExAmp) techniques. Multiple repeats of a target sequence can be present in a single nucleic acid molecule, such as a concatemer created using a rolling circle amplification procedure. Such clusters may be characterized by a degree or ratio of monoclonality, or polyclonality.

In some implementations, the nucleic acid is an oligonucleotide primer. As used herein, the term “primer” is defined as a single stranded nucleic acid sequence (e.g., single strand DNA). Some primers, referred to herein as sequencing primers, serve as a starting point for DNA synthesis. Other capture primers are part of a primer set, which serve as a starting point for template amplification and cluster generation The primer length can be any number of bases long and can include a variety of non-natural nucleotides. In an example, the sequencing primer is a short strand, ranging from 10 to 60 bases, or from 20 to 40 bases.

In embodiments in which the bioadhesive is a peptide mimetic adhesive, the 3′ or 5′ terminus of a capture primer of primer set herein may be modified to allow a coupling reaction between the capture primer and peptide to form a peptide-oligo conjugate. By way of example, the capture primer include a linker at the 3′ or 5′ terminus, in the linker is adapted to covalently bind a peptide mimetic adhesive herein to form a peptide-oligo conjugate. Such linkers may include, without limitation, thiols (e.g., Maleimide thiol), N-Acylphosphoramidate, amide, oxime, and/or urea. In other such embodiments, the side chain hydroxyl group of the peptide adhesive is modified to include serine, threonine, or tyrosine, each of which may link to the 5′-terminus of the oligonucleotide by a phosphonate diester linkage to form a peptide-oligo conjugate.

The term “sequence” in this context includes or represents a strand of nucleotides coupled to each other. The nucleotides may be based on DNA or RNA. It should be understood that one sequence may include multiple sub-sequences. For example, a single sequence (e.g., of an amplicon) may have 350 nucleotides. The sample read may include multiple sub-sequences within these 350 nucleotides. For instance, the sample read may include first and second flanking subsequences having, for example, 20-50 nucleotides. The first and second flanking sub-sequences may be located on either side of a repetitive segment having a corresponding sub-sequence (e.g., 40-100 nucleotides). Each of the flanking sub-sequences may include (or include portions of) a primer sub-sequence (e.g., 10-30 nucleotides). For ease of reading, the term “sub-sequence” will be referred to as “sequence,” but it is understood that two sequences are not necessarily separate from each other on a common strand. To differentiate the various sequences described herein, the sequences may be given different labels (e.g., target sequence, primer sequence, flanking sequence, reference sequence, and the like). Other terms, such as “allele,” may be given different labels to differentiate between like objects.

The term “read” refers to a collection of sequence data that describes a fragment of a nucleotide sample or reference. The term “read” may refer to a sample read and/or a reference read. Typically, though not necessarily, a read represents a short sequence of contiguous base pairs in the sample or reference. The read may be represented symbolically by the base pair sequence (in ATCG) of the sample or reference fragment. It may be stored in a memory device and processed as appropriate to determine whether the read matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that may be used to identify a larger sequence or region, e.g., that may be aligned and specifically assigned to a chromosome or genomic region or gene.

In some embodiments, the process to determine the nucleotide sequence of a target nucleic acid may be an automated process using an SBS technique. SBS techniques generally involve the enzymatic extension of a nascent nucleic acid strand through the iterative addition of nucleoside monomers against a template strand. SBS in general involves the enzymatic extension of a nascent copy strand through iterative addition and simultaneous detection of nucleoside monomers against a template strands. For each iteration (or cycle), labeled nucleoside monomers are detected through induced fluorescence as each monomer is added to the copy strand then replaced in a reverse-terminator process with non-labeled analogues before the start of a subsequent cycle. According to the example method, at a first cycle, (1) a buffer solution containing a mixture of four different labeled nucleoside derivatives (one for each of the four DNA nucleobases (A, G, C, T)) is provided to a cluster of template strands in the presence of polymerase; (2) a complementary nucleoside derivative is added to a nascent copy strand hybridized to each template strand via polymerase primer extension; (3) the added nucleoside derivatives are irradiated with incident light via an excitation source to induce fluorescence and emission of an output signal from the cluster; (4) the output signal is detected by one or more optical sensors through imaging of the signal as a point source at an addressed location on the optic substrate; and (5) information imparted in the imaged output signal is processed by a signal processor to record the nucleobase of the added nucleoside derivative and an address of the cluster on the optic substrate. Steps (1)-(5) are performed simultaneously for a plurality of clusters with a given frame (or tile) of the one or more optic sensors. Steps (1)-(5) are then repeated in subsequent cycles to n number of total cycles, where n is equal to the read length of the template strands in base pairs (bp).

Reads in the range of 50-100 bp may be obtained using an SBS-based technique paired with a single end sequencing chemistry, in which template strands are sequenced in one direction. Larger reads in the range of ~300 to 800 bp may be obtained using an SBS-based technique with a paired-end sequencing chemistry to generate paired-end reads of each fragment in both forward and reverse directions. Thus, for example, continuous reads may be generated for 300 bp fragments using a 150 bp cycle kit, for 600 bp fragments using a 300 bp cycle kit, and so on. Still larger reads may be generated through computational leveraging. For example, reads may be generated for 800 bp fragments using a 300 bp cycle kit by inserting a known length between the paired ends (for simplicity, a 200 bp insert corresponding to the delta between the 800 bp fragment and the 2×300 bp paired end reads) and inferring the sequence of the insert from the intersection of aligned read data in a pileup format. In one example, long insert paired-end reads are generated in combination with short insert paired reads sequenced at higher depth to infer long insert sequences.

Still larger reads in the range of several kilobases may be obtained using an SBS-based technique paired with mate pair sequencing chemistry. Here, the sample gDNA may first be tagmented at desired fragment lengths with a Mate Pair Tagment Enzyme, which attaches a biotinylated junction adapter to each end of the tagmented molecule. The tagmented DNA molecules may then be circularized and the ends of the genomic fragment linked by the respective biotin junction adapters. Circularized molecules may then be re-fragmented yielding smaller fragments suitable for amplification and sequencing. Sub-fragments containing the original junction may then be enriched via the biotin tag in the junction adapter. After End Repair and A-tailing, DNA adapters are then added, enabling amplification and sequencing. The short, fragmented reads may then be aligned to yield a long read for the tagmented fragment.

Long reads methodologies for use herein may also include linked reads (~2-5 kb) obtained using a mate-pair sequencing process (Illumina, Inc.), circular consensus sequencing (CCS) reads (~13-16 kb) using a HiFi sequencing process (PacBio), or continuous reads (~30-40 kb) using a nanopore sequencing process (Oxford Nanopore Technologies (ONT)).

SBS may utilize nucleotide monomers that have a terminator moiety or those that lack any terminator moieties. Methods utilizing nucleotide monomers lacking terminators include, for example, pyrosequencing and sequencing using γ-phosphate-labeled nucleotides. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator may be effectively irreversible under the sequencing conditions used as is the case for traditional Sanger sequencing which utilizes dideoxynucleotides, or the terminator may be reversible.

As used herein, the term “flow cell” is intended to mean a vessel having a flow channel that is in fluid communication with at least one unmodified surface or at least one surface modified with a first member of a transition metal complex binding pair. The unmodified or modified surface is capable of attaching surface chemistry that to be used in during a nucleic acid analysis and is capable of releasing the surface chemistry either electrochemically or upon exposure to visible light. The flow cell also includes an inlet for delivering reagent(s) to the flow channel and an outlet for removing reagent(s) from the flow channel. The flow cell enables the detection of the reactions involving the surface chemistry. For example, the flow cell may include one or more transparent surfaces, which allow for the optical detection of arrays, optically labeled molecules, or the like within the flow channel.

A polymeric hydrogel as referred to herein may be any gel material that can swell when liquid is taken up and can contract when liquid is removed, e.g., by drying. The hydrogel may be synthesized from natural polymers, synthetic polymers, polymerizable synthetic monomers, or a combination of natural and synthetic polymers. The hydrogel according to examples can be a poly(acrylamide), poly(methacrylate), poly(acrylate), poly(methacrylamide), polyethylene glycol, and/or polypropylene glycol.

To ensure successful sequencing, e.g., in multi-cycle SBS, the capture primers must be attached to a solid support surface in a robust manner to maintain cluster integrity and to mitigate any stressors that occur during SBS. Conventional technologies achieve this by having capture primers grafted to a polymeric hydrogel that acts as a scaffold for attachment to the solid support.

The current hydrogel deposition method, e.g., for glass flow cells, occurs via solution deposition method and then a subsequent grafting of primers to the hydrogel later in the process. The current method predominantly relies on interactions between the hydrogel and surface to drive flow cell surface adhesion. The present systems, methods, kits and devices utilizes adhesion primed hydrogels during the solution deposition to ensure robust attachment of capture primers though multiple cycles of use. The present technique offers the ability to improve surface:hydrogel adhesion by functionalizing the solid support with a polymeric hydrogel grafted with a adhesion promoting biopolymer that can directly interact both with the solid support surface and capture primers seeded to the surface for robust adhesion of the capture primers to functionalized portion of the solid support.

In that end, a functionalized solid support surface is provided in which a hydrogel adhered to the surface contains one or more adhesion promoting biopolymers. In some embodiments, the adhesion promoting biopolymer can be an oligonucleotide or oligonucleotide mixture. By way of example, each such oligonucleotide or respective oligonucleotide in a mixture can include one of following nucleobase sequences:

(SEQ ID No: 1)
TTTTTTTTTTAATGATACGGCGACCACCGAGAUCTACAC,
(SEQ ID No: 2)
TTTTTTAATGATACGGCGACCACCGAGA/vinyl-T/CTACAC,
and
(SEQ ID NO: 3)
TTTTTTTTTTCAAGCAGAAGACGGCATACGAGAT.

The adhesion promoting biopolymer consistent with the disclosure can also be an amino acids. By way of example, peptides herein may include post-translationally modified amino acids or sequence mixtures of amino acids. According to certain examples the peptide is a biomimetic peptide. Such biomimetic peptides can include (1) polydopamine, (2) domains of poly-tyrosine with post-translational modification from tyrosine to 3,4-dihydroxyphenylalanine (also known as L-DOPA, and abbreviated as “Y*”), and/or (3) repeat units of Y*-X, where X is a cationic amino acid such as lysine, arginine, or histidine. In yet other examples, the adhesion promoting biopolymer consists of a mixture of adhesion peptides and oligos.

By improving surface adhesion via biopolymer adhesives herein, a solid support system may improve output by: (a) minimizing data quality defects i.e. padhopping/duplicates or data loss due to poor surface adhesion and/or (b) improving the extensibility for long reads/cycles by increasing surface attachment robustness. Further, the present biopolymer adhesive technology is fully compatible in current flow cell fabrication processes and sequencing workflows. Thus, the present technology enables improved adhesion through multiple cycles of use while limiting costs given the technologies reliance on conventional fabrication and sequencing workflows.

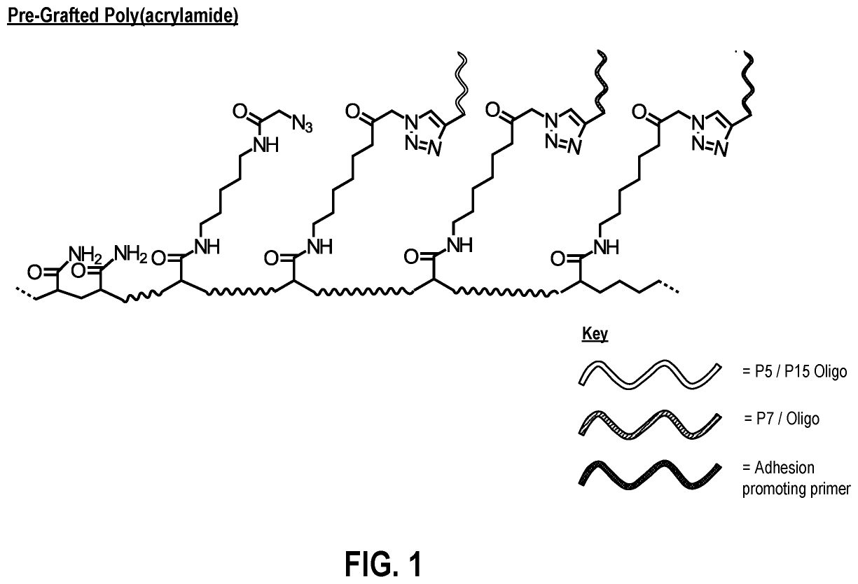

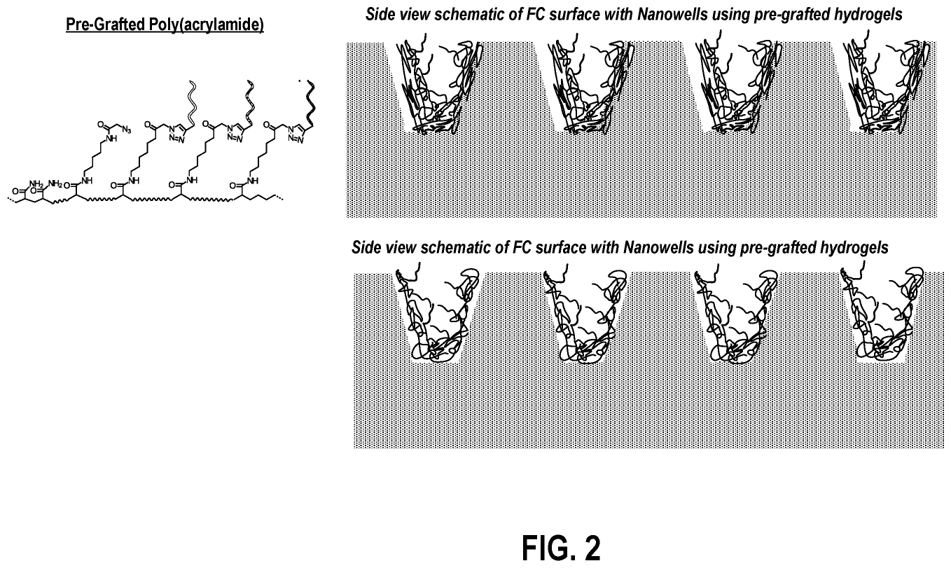

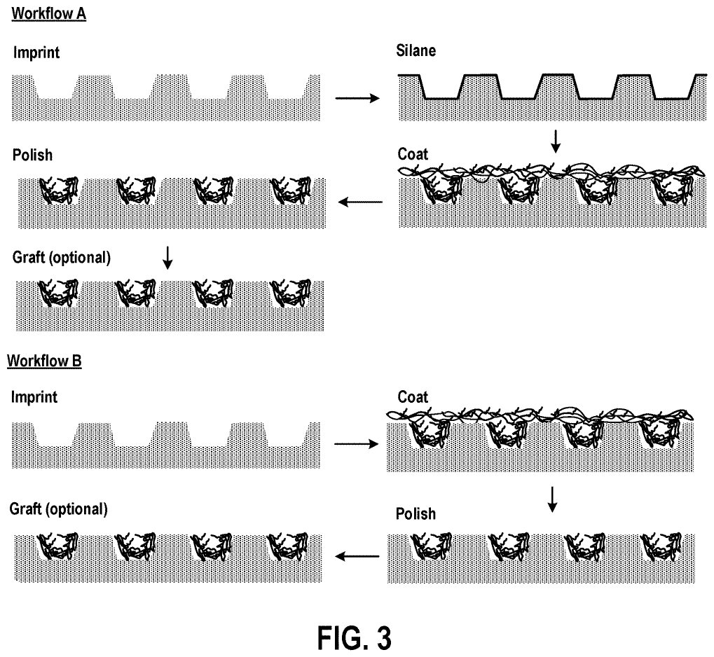

FIGS. 1-3 provide illustrated schematics of fabrication processes for use consistent with the present technology. In certain examples, as illustrated in FIGS. 2 and 3, the hydrogel—an example poly(acrylamide)—is pre-grafted with one or more adhesion promoting capture primers “off-surface,” i.e., prior to depositing or coating the hydrogel on the solid support surface. In the alternative, the one or more capture primers is grafted to the hydrogel “on surface,” i.e., after the hydrogel has been deposited or coated to the solid support surface. FIG. 1 illustrates an example of an adhesion promoting capture primer pre-grafted to poly(acrylamide) hydrogel, in which the capture primer may be covalently bound to the hydrogel in the same manner as standard p5/p7/p15 adaptor primers. In addition to covalent binding, other number of schemes may be utilized to graft the adhesion promoting capture primer to a hydrogel, including 1) hydrogen bonding, 2) Pi-Pi stacking, 3) hydrophobic interactions, 4) Van der Waals/electrostatics interactions.

FIG. 2 presents a cross-sectional illustration of an example patterned flow cell having an array of nanowells separated by interstitial regions of planar, non-reactive substrate, in which a hydrogel—either pre-grafted with adhesion capture primers (top) or non-pre-grafted (bottom)—is coated to the surface of respective nanowells. To introduce the polymeric hydrogel into the nanowells, a mixture of the polymeric hydrogel may be generated and then applied to solid support surface In one example, the pre-grafted polymeric hydrogel may be present in a mixture (e.g., with water or with ethanol and water). The mixture may then be applied to the substrate surface using spin coating, or dipping or dip coating, or flow of the material under positive or negative pressure, or another suitable technique.

In the illustration of FIG. 2, the hydrogel is coated to the surface using a conventional spin-coating technique. Generally, these types of techniques blanketly deposit the polymeric hydrogel in the nanowells and on the interstitial regions. Other selective deposition techniques (e.g., involving a mask, controlled printing techniques, etc.) may be used to specifically deposit the polymeric hydrogel in the nanowells and not on the interstitial regions.

The instantly disclosed technology is applicable to a variety of solid support systems for use in analyzing a nucleic acid, including in the context of analyzing a nucleic acid sample in a variety of fluorescent-, chemiluminescent-, colorimetric-, electrochemical-, or electrooptic-based assays In some embodiments, the functionalized solid support surfaces herein can be supported on a microfluidic architecture such as flow cells, microarrays, and integrated microfluidic circuits or imaging-generating chips for use, e.g., with solid state imaging systems, including complementary metal oxide semiconductor (CMOS) and charge-coupled device (CDD) sensor systems.

Flow cells of the illustrated embodiments provide a convenient format for supporting sequencing operations as contemplated herein, including, in particular, surface chemistries involved in the capture, amplification, and imaging of analyte constituents of a biological sample on a flow cell surface, including imaging nucleic acid fragments of a sample DNA or RNA library. For example, an NGS workflow typically employs patterned flow cells etched with billions of nanowells at fixed locations for clustering clonal populations of discrete nucleic acid fragments in individual nanowells. Such flow cells may include single-surface or multi-surface architectures. A multi-surface flow cell may include a first support surface and a second support surface, where each surface supports a pattern of nanowells for clustering clonal populations of discrete nucleic acid fragment. Appropriate structures, constructs, and imaging methods for implementing a multi-surface flow are disclosed in U.S. Pat. No. 8,039,817, which is incorporated as if set forth fully herein. By way of example, the present technology may be used in the context of the optical detection, characterization, and/or identification of nucleic acid materials. In certain embodiments, the functionalized solid support surface can be incorporated in a biological assay useful for the detection of a nucleic acid sample or constituent analyte(s) thereof, in which the functionalized solid support surface is seeded with an oligonucleotide primer configured to hybridize to a target nucleic acid material or constituent analyte, and wherein each primer is attached to a respective adhesion promoting biopolymer of the hydrogel. In one example, a biological assay implemented on a functionalized solid support surface herein can be utilized in a method for optical detection of a nucleic acid material or one or more of constituent analytes. The method can include providing an optical detection system comprising an excitation source, one or more optical sensors, and a signal processor. In example embodiments, the method can further include irradiating, via the light source, at least a portion of the functionalizes support surface of the biological assay with an incident light; detecting, via the one or more optical sensors, an output signal emitted by the nucleic acid sample or constituent analyte(s) as an optic field response to the incident light, and obtaining from the output signal, via the signal processor, data indicative of a characteristic of the sample or constituent analyte(s).

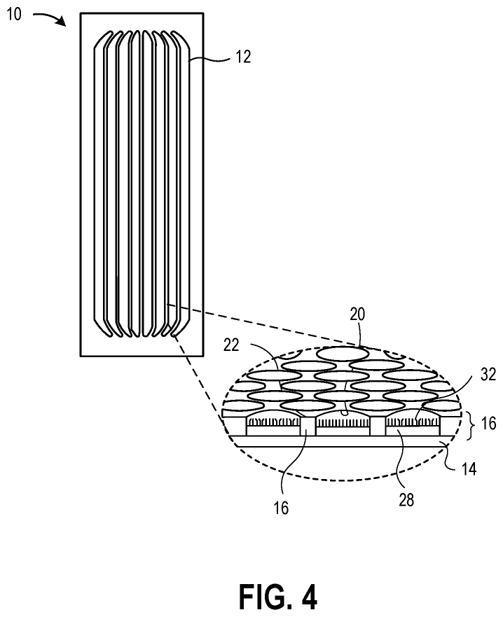

FIG. 3 illustrates fabrication workflows A and B for functionalizing a flow cell or other solid support system: workflow A including a silanization step and workflow B without a silanization step. One example of supported flow cell architecture appropriate for use herein is the flow cell 10 as shown in FIGS. 4A and 4B. The example shown in FIG. 4A includes eight flow channels 12. While eight flow channels 12 are shown, it is to be understood that any number of flow channels 12 may be included in the flow cell 10 (e.g., a single flow channel 12, four flow channels 12, etc.).

Generally, as shown in FIG. 4B, flow cell 10 may include a patterned structure, e.g., an array of nanowells 20 separated by interstitial regions 22 of planar, nonfunctionalized flow cell substrate. As further illustrated, the patterned structure may be organized into lanes 12, each separated by non-patterned, non-functionalized barrier regions, which may be bonded to a lid 20 to form flow channels 12 along each lane of patterned structure.

As illustrated in FIG. 4B, the flow channel 12 may include a multi-layered or composite structure 18, which includes, a minimum, a single layer base support 14 overlayed with a resin or other appropriate film layer. In an example, the single base support 14 (whether used singly or as part of the multi-layered structure) may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (~3 meters). For example, a die may have a width ranging from about 0.1 mm to about 10 mm. While example dimensions have been provided, it is to be understood that a single base support 14 with any suitable dimensions may be used.

The support layer may be any suitable low-background material, including materials exhibiting both high transmissivity and high fluorescence transparency, particularly for use as solid supports for fluorescence-based imaging implementations. Examples of suitable single layer base supports 14 include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, silica-based materials, aluminum silicate, silicon and modified silicon (e.g., boron doped p+ silicon), silicon nitride (Si3N4), silicon oxide (SiO2), tantalum pentoxide (Ta₂O₅) or other tantalum oxide(s) (TaOₓ), hafnium oxide (HfO₂), carbon, metals, inorganic glasses, or the like. In one example, single base support layer 14 is a glass, for example, alkaline earth boro-aluminosilicate glass (e.g., EAGLE XG® (Corning, NY)), which has an annealing point (1013 poises) rated ~1332° F.

The solid support layer on which a nucleic acid sample is detected and/or analyzed can be composed of any low-background, reagent resistant material suitable for use in analyzing a nucleic acid sample in a fluorescent-, chemiluminescent-, colorimetric-, electrochemical-, or electrooptic-based assays, including polymeric resins, epoxy silane, glass, silicone or silicone polymers (poly-dimethylsiloxane (PDMS)), plastics (e.g., acrylics, polystyrene, cyclic olefins), polyamides, polycarbonates, polyimides, silica, and the like, or any appropriate high-index, dielectric materials for use in photonic or plasmonic-based assays, including, e.g, metal oxides, nitrides or phosphides, transition metals, ceramics, and the like.

In the illustrated embodiment, an optic support layer 16 may be applied to the base support layer 14 and then patterned. Some examples of suitable resins include a polyhedral oligomeric silsesquioxane resin (POSS)-based resin, a non-POSS epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. Generally, the optic support layer 16 (or other solid support layer) have sufficient thickness to accommodate the depth of nanowells 20, which can range, for example, between 0.1 nm to 1 μm or 0.01 nm to 600 nm depending on the application. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc. In one example (shown in FIG. 4B), the thickness of the optic support layer 16 may be coterminous with the depth of nanowell 20, such that a base portion of the inner surface of nanowell 20 exposes a surface of the single layer base supports 14, in which case the single layer base support forms the solid support surface. Alternatively, the thickness of the optic support layer may accommodate the entire depth of nanowell 20, such the entire inner surface of nanowell 20 is formed in the material of the optic support layer, in which case the optic support layer 16 forms the solid support surface.

In some embodiments, a metal oxide may be selectively applied to the base support 14 as the solid support surface. The metal oxide may be applied using a number of techniques, including via vapor deposition, aerosol printing, or inkjet printing. Examples of suitable inorganic oxides include, e.g., tantalum oxide (e.g., Ta₂O₅), aluminum oxide (e.g., Al₂O₃), hafnium oxide (e.g., HfO₂), etc.

Referring back to FIG. 3, in some examples, the solid support surface may first be activated before applying the polymeric hydrogel 28. In the example workflow A of FIG. 3, a silane or silane derivative (e.g., norbornene silane) may be deposited on the solid support surface using vapor deposition, spin coating, or other deposition methods. In another example, the solid support surface may be exposed to plasma ashing to generate surface-activating agent(s) (e.g., —OH groups) that can adhere to the polymeric hydrogel 28 once applied. In the example of workflow B, the activating step of adding silane (or providing other activation) is eliminated, which may offer extensibility for different type workflows.

Again, to introduce the polymeric hydrogel (polymeric hydrogel 28, FIG. 4B) into the nanowells, a mixture of the polymeric hydrogel may be generated and then applied to solid support surface (e.g., with water or with ethanol and water). The mixture may then be applied to the substrate surface using spin coating, or dipping or dip coating, or flow of the material under positive or negative pressure, or another suitable technique. Depending upon the chemistry of the polymeric hydrogel, the applied mixture may be exposed to a curing process. In an example, curing may take place at a temperature ranging from room temperature (e.g., about 25° C.) to about 95° C. for a time ranging from about 1 millisecond to about several days.

Still referring to the workflows of FIG. 3, after coating the solid support surface with the polymeric hydrogel, polishing may then be performed in order to remove the polymeric hydrogel from the interstitial regions (see interstitial regions 22, FIG. 4B), while leaving the polymeric hydrogel on the surface in the nanowells at least substantially intact.

Optionally, the adhesive capture primers can be pre-grafted to the polymeric hydrogel prior coating. Otherwise, as illustrated in FIG. 3 workflows, the adhesive capture primers 32 may be grafted onto hydrogel coating in a reagent flowed along the solid support surface.

### Example 1—Debond Shear Stress Test of Pre-Grafted Hydrogel Surface Adhesion

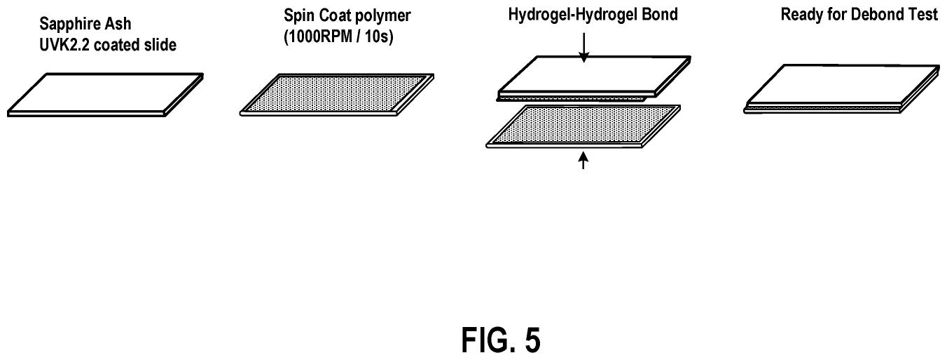

With reference to FIG. 5, to test the adhesive strength of pre-grafted polymers of Table 1, two glass slides were bonded and adhesive forces were measured with a debond shear stress test.

TABLE 1
Adhesion Promoting Oligo Capture Primers

| Name | Details | Class |
|---|---|---|
| P5 | Sequence: | Nucleobase |
| | TTTTTTTTTTAATGATACGGCGACCACCGA | |
| | GAUCTACAC (SEQ ID NO: 1) | |
| P15 | Sequence: | Nucleobase |
| | TTTTTTAATGATACGGCGACCACCGAGA/ | |
| | vinyl-T/CTACAC (SEQ ID NO: 2) | |
| P7 | Sequence: | Nucleobase |
| | TTTTTTTTTTCAAGCAGAAGACGGCATACG | |
| | AGAT (SEQ ID NO: 3) | |

Here, a glass slide was ashed and the polymeric hydrogel (Pre-grafted P5/P7/P15 and ungrafted) was spin coated onto the surface. A second coated slide was fabricated with the above-mentioned method and the two slides were bonded together under load for 10 minutes and left at room temperature for 3 days. The bonded glass slide were then loaded into a debond shear test to measure the force required to de-bond the slides.

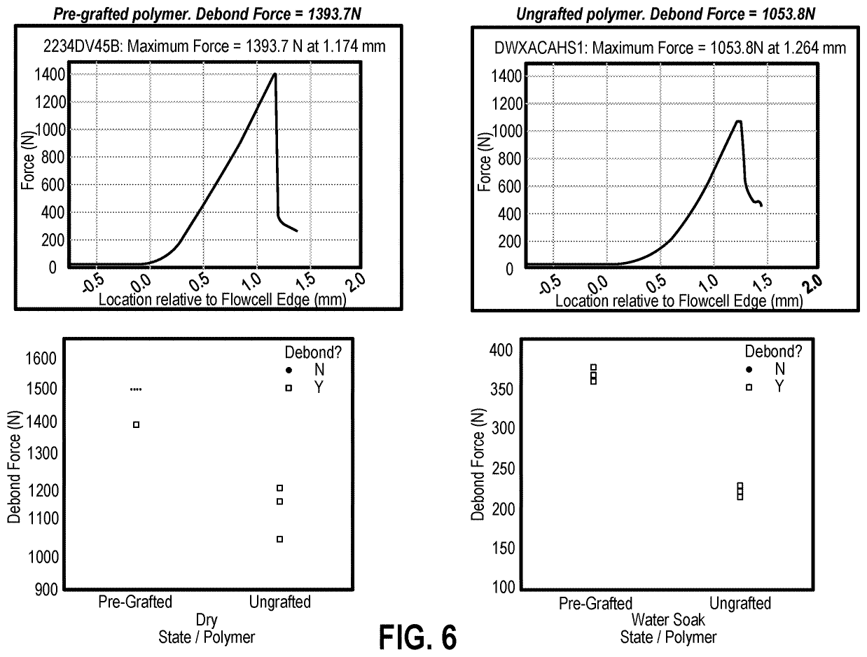

With reference to FIG. 6, comparing the force required for debond shows a notable increase in adhesive strength, the ungrafted hydrogel gave debond values ~1100N whereas pre-grafted hydrogel gave values >~1400N and in the majority of cases no debonding occurred for pre-grafted polymers. (Note debond tool limit is 1500N.) Furthermore, samples were stored in water for 36 hrs and tested for debond strength, similar to the dry samples a pre-grafted polymer shows higher debond force values. The higher values indicate that the pre-grafted hydrogels have significantly increased adhesion, and this will is apparent in fluidic environments.

In one example, functionalized solid support surfaces described herein may be used in a method for optical detection of a nucleic acid sample in a sequencing protocol is provided. Here, flow cells provide a convenient format in the example sequencing protocol, which can involve multiple cycles of repeated chemical delivery and image capture. According to the example method, a DNA sample (e.g., gDNA) or an RNA sample (e.g., as cDNA) in the form of a DNA library can be obtained in which the DNA sample is processed into constituent fragment strands and ligated with technology-specific adaptors. According to the example method, the DNA library can be loaded onto an optic support layer of a flow cell device, where constituent fragment strands are flowed across an array of nanowells impressed in the optic support layer and individual strands are absorbed by nanowells of the array at a substantially 1:1 basis, where each absorbed strand is immobilized at a reaction site of respective nanoantennae through interaction between the strand adaptor and a capture primer covalently bound to the surface of the reaction site. Each immobilized strand can then be amplified (e.g., using bridge or exclusion amplification) to yield a substantially monoclonal cluster of template strands within each respective nanoantennae.

Each of the resulting clusters is then sequenced using, e.g., an SBS technique. SBS in general involves the enzymatic extension of a nascent copy strand through iterative addition and simultaneous detection of nucleoside monomers against a template strands. For each iteration (or cycle), nucleoside monomers labeled with a fluorescent species can be detected through induced fluorescence as each monomer is added to the copy strand then replaced in a reverse-terminator process with non-labeled analogues before the start of a subsequent cycle. According to the example method, in a first cycle, (1) a buffer solution containing a mixture of four different labeled nucleoside derivatives (one for each of the four DNA nucleobases (A, G, C, T)) can be provided to a cluster of template strands in the presence of polymerase; (2) a complementary nucleoside derivative can be added to a nascent copy strand hybridized to each template strand via polymerase primer extension; (3) the added nucleoside derivatives can be irradiated with incident light via an excitation source to induce fluorescence and emission of an output signal from the cluster; (4) the output signal can be detected by one or more optical sensors through imaging of the signal as a point source at an addressed location on the optic substrate; and (5) information imparted in the imaged output signal can be processed by a signal processor to record the nucleobase of the added nucleoside derivative and an address of the cluster on the optic substrate. Steps (1)-(5) can be performed simultaneously for a plurality of clusters with a given frame (or tile) of the one or more optic sensors. Steps (1)-(5) can then be repeated in subsequent cycles to n number of total cycles, where n is equal to the size of the template strands in base pairs (bp).

Here, the flow cell surface may have a layout or pattern of nanowells that may be characterized with respect to the density (number), porosity (pore area as a % portion of film area), pitch (center-point distance between neighboring wells of nanowells 20 in nm), nanowell diameter (nm), and nanowell depth (nm) of the nanowells 20 in a defined area.

For example, referring again to FIG. 4B, the nanowells 20 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high-density array may be characterized as having the nanowells 20 separated by less than about 100 nm, a medium density array may be characterized as having the nanowells 20 separated by about 400 nm to about 1 μm, and a low-density array may be characterized as having the nanowells 20 separated by greater than about 1 μm.

The layout or pattern of the nanowells 20 may also or alternatively be characterized in terms of the average pitch, or the spacing from the center of one nanowell 20 to the center of an adjacent nanowell 20 (center-to-center spacing) or from the right edge of one nanowell 20 to the left edge of an adjacent nanowell 20 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern can be between one of the lower values and one of the upper values selected from the ranges above. In some embodiments, the nanowells 20 are nanowells and have an average pitch (center-to-center spacing) about 250 nm or greater, 300 nm or greater, 350 nm or greater, 400 nm or greater, 450 nm or greater, 500 nm or greater, 550 nm or greater, 600 nm or greater, 650 nm or greater, or 700 nm or greater, or may be in a range between about 250 nm and 800 nm, 300 nm and 750 nm, 350 nm and 700 nm, 400 nm and 650 nm, 450 nm and 600 nm, 500 nm and 550 nm. In an example, the nanowells 20 are nanowells and have an average pitch (center-to-center spacing) between about 350 nm and 750 nm. While example average pitch values have been provided, it is to be understood that other average pitch values may be used.

The nanowells 20 may be characterized by the geometric shape of a cross-section of the nanowell 20 taken parallel to a predetermined plane, such as a face of the base support 22, or by the volume, opening area, depth, diameter, length, or width of the nanowell 20, or by a combination thereof. For example, the nanowells 20 may be nanowells with a hexagonal morphology, or the nanowells 20 may be nanowells with hexagonal openings, or the nanowells 20 may be nanowells configured cylindrical structures with substantially circular openings. For another example, the opening area can range from about 1×10-3 μm² to about 100 μm², e.g., about 1×10-2 μm², about 0.1 μm², about 1 μm², at least about 10 μm², or more, or less. In another example, the volume can range from about 1×10-3 μm³ to about 100 μm³, e.g., about 1×10-2 μm³, about 0.1 μm³, about 1 μm³, about 10 μm³, or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less. In another example, the nanowells 20 have an average depth of 150 nm or greater, 200 nm or greater, 250 nm or greater, 300 nm or greater, 350 nm or greater, or 400 nm or greater, or may be in a range between about 150 nm and 500 nm, 200 nm and 450 nm, or 250 nm and 400 nm, or 300 nm and 350 nm.

The depth of the flow channel 12 can be as small as a monolayer thick when microcontact, aerosol, or inkjet printing is used to deposit a separate material over the bonding region 26 that defines the flow channel 12 walls. In other examples, a thicker spacer layer may be applied to bonding region 26 so that the spacer layer defines at least a portion of the walls of the flow channel 12. As one example, the spacer layer can be a radiation-absorbing material that aids in bonding. In these examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than or between the values specified above.

Fluorescent species for use consistent with SBS and other optical detection methodologies may be any target analyte, analyte conjugate, or other moiety that can be detected based on an optical field response to excitation. Fluorescing species may include a target analyte with inherent fluorescence (e.g., peptide tracers) or detectible label moieties. Exemplary labels for use consistent with various embodiments, for example, include chromophores; luminophores; fluorophores; optically encoded nanoparticles; particles encoded with a diffraction-grating; electrochemiluminescent labels such as Ru(bpy).sup.32+; or other species capable of detection based on an optical characteristic. In the instant disclosure the conjugated polymer fluorophores described herein are useful as a fluorescent species. Other fluorophores that may be useful include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dyes, phycoerythin, bodipy, and others known in the art.

The density of the patterned array may also be understood in terms of the number of fluorescing analytes present per unit area. For example, the average density of analytes for an array may be at least about 1×10³ analytes/mm², 1×10⁴ analytes/mm², 1×10⁵ analytes/mm², 1×10⁶ analytes/mm², 1×10⁷ analytes/mm², 1×10⁸ analytes/mm², or 1×10⁹ analytes/mm², or higher. Alternatively, or additionally the average density of analytes for an array may be at most about 1×10⁹ analytes/mm², 1×10⁸ analytes/mm², 1×10⁷ analytes/mm², 1×10⁶ analytes/mm², 1×10⁵ analytes/mm², 1×10⁴ analytes/mm², or 1×10³ analytes/mm², or less.

The size and shape of analytes in a pattern may be determined by the size and shape of nanostructures in the functionalized solid support. For example, when observed in a two-dimensional plane, such as on the surface of support, the analytes may appear rounded, circular, oval, rectangular, square, symmetric, asymmetric, triangular, polygonal, or the like. The analytes may be arranged in a regular repeating pattern including, for example, a hexagonal or rectilinear pattern. A pattern may be selected to achieve a desired level of packing. For example, round analytes are optimally packed in a hexagonal arrangement. Of course, other packing arrangements may also be used for round analytes and vice versa.

A pattern may be characterized in terms of the number of analytes that are present in a subset that forms the smallest geometric unit of the pattern. The subset may include, for example, at least about 2, 3, 4, 5, 6, 10 or more analytes. Depending upon the size and density of the analytes the geometric unit may occupy an area of less than 1 mm², 500 μm², 100 μm², 50 μm², 10 μm², 1 μm², 500 nm², 100 nm², 50 nm², 10 nm², or less. Alternatively, or additionally, the geometric unit may occupy an area of greater than 10 nm², 50 nm², 100 nm², 500 nm², 1 μm², 10 μm², 50 μm², 100 μm², 500 μm², 1 mm², or more. Characteristics of the analytes in a geometric unit, such as shape, size, pitch, and the like, may be selected from those set forth herein more generally with regard to analytes in an array or pattern.

An functionalized solid support having a regular pattern of analytes may be ordered with respect to the relative locations of the analytes but random with respect to one or more other characteristic of each analyte. For example, in the case of a nucleic acid array, the nuclei acid analytes may be ordered with respect to their relative locations but random with respect to one's knowledge of the sequence for the nucleic acid species present at any particular analyte. As a more specific example, nucleic acid arrays formed by seeding a repeating pattern of analytes with template nucleic acids and amplifying the template at each analyte to form copies of the template at the analyte (e.g., via cluster amplification, bridge amplification, or exclusion amplification (ExAmp)) will have a regular pattern of nucleic acid analytes but will be random with regard to the distribution of sequences of the nucleic acids across the array. Thus, detection of the presence of nucleic acid material generally on the array may yield a repeating pattern of analytes, whereas sequence specific detection may yield non-repeating distribution of signals across the array.

The technology described herein may be used in conjunction with a variety of nucleic acid sequencing techniques. Particularly applicable techniques are those in which nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are also applicable.

Reads in the range of 50-100 bp may be obtained using an SBS-based technique paired with a single end sequencing chemistry, in which template strands are sequenced in one direction. Larger reads in the range of ~300 to 800 bp may be obtained using an SBS-based technique with a paired-end sequencing chemistry to generate paired-end reads of each fragment in both forward and reverse directions. Thus, for example, continuous reads may be generated for 300 bp fragments using a 150 bp cycle kit, for 600 bp fragments using a 300 bp cycle kit, and so on. Still larger reads may be generated through computational leveraging. For example, reads may be generated for 800 bp fragments using a 300 bp cycle kit by inserting a known length between the paired ends (for simplicity, a 200 bp insert corresponding to the delta between the 800 bp fragment and the 2×300 bp paired end reads) and inferring the sequence of the insert from the intersection of aligned read data in a pileup format. In one example, long insert paired-end reads are generated in combination with short insert paired reads sequenced at higher depth to infer long insert sequences.

Still larger reads in the range of several kilobases may be obtained using an SBS-based technique paired with mate pair sequencing chemistry. Here, the sample gDNA may first be tagmented at desired fragment lengths, which attaches a biotinylated junction adapter to each end of the tagmented molecule. The tagmented DNA molecules may then be circularized and the ends of the genomic fragment linked by the respective biotin junction adapters. Circularized molecules may then be re-fragmented yielding smaller fragments suitable for amplification and sequencing. Sub-fragments containing the original junction may then be enriched via the biotin tag in the junction adapter. After end Repair and A-tailing, DNA adapters are then added, enabling amplification and sequencing. The short, fragmented reads may then be aligned to yield a long read for the tagmented fragment.

SBS may utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator may be effectively irreversible under the sequencing conditions used as is the case for traditional Sanger sequencing which utilizes dideoxynucleotides, or the terminator may be reversible.

As one example, SBS reaction may be run on any SBS system platform. In SBS, extension of a nucleic acid primer (e.g., a sequencing primer) along a nucleic acid template (i.e., the sequencing template) is monitored to determine the sequence of nucleotides in the template. The underlying chemical process can be polymerization (e.g., catalyzed by a polymerase enzyme) or ligation (e.g., catalyzed by a ligase enzyme). In a particular polymerase-based SBS process, fluorescently labeled nucleotides are added to the sequencing primer (thereby extending the sequencing primer) in a template dependent fashion such that detection of the order and type of nucleotides added to the sequencing primer can be used to determine the sequence of the template.

Sequencing methodologies for nucleic acid samples on NGS platforms commonly deploy DNA libraries in which a DNA target (e.g., genomic DNA (gDNA), or complimentary DNA (cDNA)) is processed into fragments and ligated with technology-specific adaptors. Fragments may also be ligated with sample source-specific barcoding in multiplexed operations. NGS workflow using SBS involves loading a DNA library onto a flow cell and hybridizing individual DNA fragments to adapter-specific complimentary oligonucleotides (i.e., capture primers) covalently bound to the flow cell surface.

In one example, a functionalized solid support surface supported on a flow cell architecture is provided in which a hydrogel adhered to the surface contains one or more adhesion promoting biopolymers. In some embodiments, the adhesion promoting biopolymer can be an oligonucleotide or oligonucleotide mixture, in which case the adhesion promoting biopolymer is adapted to hybridize to a portion of the sequencing primer. By way of example, each such oligonucleotide or respective oligonucleotide in a mixture can include one of following nucleobase sequences:

(SEQ ID No: 1)
TTTTTTTTTTAATGATACGGCGACCACCGAGAUCTACAC,
(SEQ ID No: 2)
TTTTTTAATGATACGGCGACCACCGAGA/vinyl-T/CTACAC,
and
(SEQ ID NO: 3)
TTTTTTTTTTCAAGCAGAAGACGGCATACGAGAT.

The adhesion promoting biopolymer consistent with the disclosure can also be an amino acids. By way of example, peptides herein may include post-translationally modified amino acids, sequence mixtures of amino acids, and/or post-translationally modified amino acids. According to certain examples the peptide is a biomimetic peptide. Such biomimetic peptides can include (1) polydopamine, (2) domains of poly-tyrosine with post-translational modification from tyrosine to 3,4-dihydroxyphenylalanine (also known as L-DOPA, and abbreviated as “Y*”), and/or (3) repeat units of Y*-X, where X is a cationic amino acid such as lysine, arginine, or histidine.

In the case of biomimetic peptide primers or primer sets, the 3′ or 5′ terminus of a capture primer of primer set forth herein may be modified to allow a coupling reaction between the capture primer and peptide to form a peptide-oligo conjugate. By way of example, the capture primer include a linker at the 3′ or 5′ terminus, in the linker is adapted to covalently bind a peptide mimetic adhesive herein to form a peptide-oligo conjugate. Such linkers may include, without limitation, thiols (e.g., Maleimide thiol), N-Acylphosphoramidate, amide, oxime, and/or urea. In other such embodiments, the side chain hydroxyl group of the peptide adhesive is modified to include serine, threonine, or tyrosine, each of which may link to the 5′-terminus of the oligonucleotide by a phosphonate diester linkage to form a peptide-oligo conjugate.

In an example of conjugation through the N-Acylphosphoramidate Linkage, the N-acylphosphoramidates may be prepared in two steps: (1) coupling an N-phosphitylated carboxamide with alcohol, in the presence of coupling agents such as tetrazole; and (2) subsequent oxidation of the resulting phosphite to phosphate. (Alternatively, the N-acylphosphoramidates can also be prepared by direct coupling of the phosphatylated primary carboxamide to the oligonucleotide.) The peptide and oligonucleotide fragments of these conjugates are linked through N-acylphosphoramidate functional groups.

The maleimide-thiol linkage is another commonly used strategies to generate peptide-oligo conjugates. In this strategy, oligonucleotides or peptides containing a maleimide functional group are treated with the peptides or oligonucleotides containing a thiol group, to yield the corresponding conjugates, linked through a maleimido-thioether group. The conjugation may also occur through oxime formation by treating an oxyamine containing oligonucleotide or peptide with an aldehyde containing peptide or oligonucleotide. As another example, conjugation may occur through thiazolidine formation by treating a peptide containing a cysteine residue with an oligonucleotide containing an aldehyde function.

Referring again to FIG. 4B, once individual DNA fragments are hybridized the capture primers, amplification of the template nucleic acid strand(s) may be initiated to form a cluster of the template stands across the polymeric hydrogel in each nanowell 20. In one example of cluster generation, the library fragments are copied by 3′ extension using a high-fidelity DNA polymerase. The original library fragments are denatured, leaving the copies immobilized. Isothermal bridge amplification may be used to amplify the immobilized copies. For example, the copied templates loop over to hybridize to an adjacent, complementary sequencing primer and a polymerase copies the copied templates to form double stranded bridges, which are denatured to form two single stranded strands. These two strands loop over and hybridize to adjacent, capture primers and are extended again to form two new double stranded loops. The process is repeated on each template copy by cycles of isothermal denaturation and amplification to create dense clonal clusters. Each cluster of double stranded bridges is denatured. In an example, the reverse strand is removed by specific base cleavage, leaving forward template polynucleotide strands. Clustering results in the formation of many multiple template polynucleotide strands (or amplicons) on the functionalized solid support surface of each nanowell 20.

Amplification may also be performed using an exclusion amplification (ExAmp) technique. ExAmp cluster generation is particularly appropriate for optimizing monoclonality of amplicon populations on a given functionalized solid support surface. ExAmp chemistry carries out seeding and cluster amplification steps simultaneously such that amplification of a first seeding event on a given functionalized solid support surface occurs nearly instantaneously and the amplification rate far exceeds the reaction rate for seeding. In that manner, the ExAmp amplification of a fragment seeded to a reaction site prevents further seeding by other fragments, thus reducing the occurrence of undesirable polyclonal clustering.

Some examples of the method then include blocking non-protected (free) 3′ OH ends of the template strands and primers that do not have template strands attached thereto. A blocking group (e.g., a 3′ phosphate) may be added that attaches to the exposed 3′ ends to prevent undesired extension.

Sequencing primers may then be introduced to the flow cell. The sequencing primers hybridize to the template nucleic acid strands. These sequencing primers render the template strands ready for sequencing. An incorporation mix including nucleotides labelled with fluorescent species may then be introduced into the flow cell, e.g., via an inlet. In addition to the labeled nucleotides, the incorporation mix may include water, a buffer, and polymerases. Referring to FIG. 1A, when the incorporation mix is introduced into the flow cell 10, the mix enters the flow channel 12, and contacts the anchored and sequence ready template strands.

The incorporation mix is allowed to incubate in the flow cell 10, and labeled nucleotides (including optical labels) are incorporated by respective polymerases into the nascent strands along the template strands. During incorporation, one of the labeled nucleotides is incorporated, by a respective polymerase, into one nascent strand that extends one sequencing primer and that is complementary to one of the template strands. Incorporation is performed in a template strand dependent fashion, and thus detection of the order and type of labeled nucleotides added to the nascent strand can be used to determine the sequence of the template strand. Incorporation occurs in at least some of the template strands across the flow cell 10 during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including nonincorporated labeled nucleotides, may be removed from the flow cell 10 during a wash cycle. The wash cycle may involve a flow-through technique, where a washing solution (e.g., buffer) is directed into, through, and then out of flow channel 12, e.g., by a pump or other suitable mechanism.

Without further incorporation taking place, the most recently incorporated labeled nucleotides can be detected through an imaging event. During the imaging event, an illumination system may provide an excitation light to the flow cell 10. The optical labels of the incorporated labeled nucleotides emit optical signals in response to the excitation light. These optical signals may be captured using an imaging device.

After imaging is performed, a cleavage mix may then be introduced into the flow cell 10. In an example, the cleavage mix is capable of i) removing the 3′ OH blocking group from the incorporated nucleotides, and ii) cleaving the optical label from the incorporated nucleotide. Examples of 3′ OH blocking groups and suitable deblocking agents/components in the cleavage mix may include: ester moieties that can be removed by base hydrolysis; allyl-moieties that can be removed with Nal, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include sodium periodate, which can cleave a vicinal diol; phosphines, such as tris(2-carboxyethyl)phosphine (TCEP) or tri(hydroxypropyl)phosphine (THP), which can cleave azidomethyl linkages; palladium and THP, which can cleave an allyl; bases, which can cleave ester moieties; or any other suitable cleaving agent of the 3′ OH blocking group.

Additional sequencing cycles may then be performed until the template strands 40 are sequenced. The nascent strands may be dehybridized, and the blocking group at the 3′ OH ends of the template strands and primers may be removed. Clustering is performed again, and this time, the forward strands are removed by specific cleavage at the cleavage site (e.g., uracil, 8-oxoguanine, allyl-T, etc. in the primer sequence, leaving the reverse template strands. Sequencing of the reverse template strands may be performed as described herein.

After sequencing, the cleaving fluid is introduced into the flow cell 10, e.g., via the inlet, to cleave the grafted plurality of primers at the thioester-cysteine bond, thereby leaving a plurality of thioester functional groups at the surface of the flow cell 10. After a desired time for cleaving, a wash cycle may be performed to remove the cleaved portions.

With multiple reactive sulfur-containing functional groups again located at the surface of the polymeric hydrogel, the flow cell surface is ready for another round of primer grafting and nucleic acid analysis. The processes may be repeated as desired to perform multiple nucleic acid analyses.

Other example sequencing techniques consistent for use with the technology herein include, e.g., pyrophosphate sequencing (e.g., Genome Sequencer FLX from 454 Life Sciences), ion-sensitive sequencing (e.g., Personal Genome Machine and Proton from Ion Torrent Systems, Inc.), Helicos True Single Molecule Sequencing (tSMS) technology, chemical-sensitive field effect transistor (chemFET) array, probe-anchor ligation sequencing (e.g., Complete GeGenomics™ or Polonator™), to name a few.

The present technology may also be utilized in probe-grafted arrays for screening nucleic acid materials for a locus of interest. Such microarrays may include deoxyribonucleic acid (DNA) or ribonucleic acid (RNA) capture probes, which are specific for nucleotide sequences present in humans and other organisms. In certain applications, for example, individual DNA or RNA probes may be grafted at addressable reaction sites on an array surface. A test sample, such as from a known person or organism, can be exposed to the array, such that target nucleic acids hybridize to complementary probes grafted on the array. The probes can be labeled in a target specific process (e.g., due to labels present on the target nucleic acids or due to enzymatic labeling of the probes or targets that are present in hybridized form). The array may then be examined by scanning specific frequencies of light over the analytes to identify which target nucleic acids are present in the sample.

By way of example, a genotyping application as contemplated may be implemented to screen for the presence of a genetic locus of interest in a target nucleic acid sample. A locus of interest in a typical genotyping protocol, and as disclosed herein, may include, without limitation, polymorphs (e.g., single nucleotide polymorphs (SNPs), indels), short tandem repeats (STR), copy number variants (CNV), germline variants, methylation sites (e.g., CpG islands), and exogenous sequences (e.g., virus). Target nucleic acid samples herein may include polynucleotides of any length and may be derived from any number of genetic sources including from human or non-human organisms, and from individual organisms or organism populations. Samples herein may be obtained from a wide variety of genetic materials—e.g., gDNA, mtDNA, mRNA, cDNA transcribed from mRNA, non-coding RNA, and small RNA, polynucleotide conjugates, analogues, and amplicons.

Image-generating chip arrays provide a convenient format for assaying SNPs, particularly at commercial scale. An example workflow may begin with accession and extraction of a DNA sample, either from single cell source or a tissue sample. The extracted DNA sample may be amplified, usually off-chip in solution, and the amplicon output is then subjected to controlled enzymatic fragmentation. The processed DNA sample is loaded onto the image-generating chip and subjected to hybridization using locus specific oligo probes functionalized on the chip substrate. Allelic specificity of hybridized DNA is conferred by enzymatic base extension at the 3′ end of the probe. Base extensions are applied fluorescent labels, imaged under excitation, and allele signal intensity data is used to perform genotype calling. An array may be functionalized with an individual probe or a population of probes. In the latter case, the population of probes at each analyte is typically homogenous having a single species of probe. For example, in the case of a nucleic acid array, each locus specific probe may be amplified to yield multiple nucleic acid molecules each having a common sequence. However, in some implementations the population of probes at a given reaction site of an array can be heterogeneous.

What is claimed is:

1. A functionalized solid support surface for detection of a polynucleotide sample,
wherein the surface comprises a polymeric hydrogel grafted with an adhesion promoting biopolymer,
wherein the adhesion promoting biopolymer is configured to covalently bind a capture primer,
wherein the capture primer is configured to hybridize with the polynucleotide sample.

2. The functionalized solid support surface of claim 2, wherein the adhesion promoting biopolymer is an oligonucleotide having one of following nucleobase sequences:
(SEQ ID No: 1)
TTTTTTTTTTAATGATACGGCGACCACCGAGAUCTACAC,
(SEQ ID No: 2)
TTTTTTAATGATACGGCGACCACCGAGA/vinyl-T/CTACAC,
and
(SEQ ID No: 3)
TTTTTTTTTTCAAGCAGAAGACGGCATACGAGAT,
wherein the adhesion promoting oligonucleotide is configured to hybridize with the capture primer, and
wherein the capture primer is configured to hybridize with the polynucleotide sample or a constituent(s) thereof.

3. The functionalized solid support surface of claim 1, wherein the adhesion promoting biopolymer comprises a peptide, and
wherein the peptide and/or capture primer is adapted to form a peptide-oligo conjugate, and
wherein the capture primer is configured to hybridize with the polynucleotide sample or a constituent(s) thereof.

4. The functionalized support surface of claim 3, wherein the peptide comprises a post-translationally modified amino acid or sequence mixtures of amino acids.

5. The functionalized solid support surface of claim 3, wherein the peptide is a biomimetic adhesive peptide.

6. The functionalized solid support surface of claim 5, wherein the biomimetic peptide comprise more or more of (1) polydopamine, (2) domains of poly-tyrosine with post-translational modification from tyrosine to 3,4-dihydroxyphenylalanine (also known as L-DOPA, and abbreviated as “Y*”), and/or (3) repeat units of Y*-X, where X is a cationic amino acid such as lysine, arginine, or histidine.

7. A biological assay for detection of a nucleic acid sample, the assay comprising
a functionalized solid support surface, wherein the solid support surface comprises a hydrogel layer adhered to the surface, wherein the hydrogel contains one or more adhesion promoting biopolymers;
a capture primer bound to the solid support surface through interaction with the one or one or more adhesion promoting biopolymers; and
a nucleic acid sample immobilized on the solid support surface via interaction with the capture primer.

8. The biological assay claim 7, wherein at least one of the one or more adhesion promoting biopolymers comprises an oligonucleotide or oligonucleotide mixture, each having a nucleobase sequences of one of:
(SEQ ID No: 1)
TTTTTTTTTTAATGATACGGCGACCACCGAGAUCTACAC,
(SEQ ID No: 2)
TTTTTTAATGATACGGCGACCACCGAGA/vinyl-T/CTACAC,
and
(SEQ ID No: 3)
TTTTTTTTTTCAAGCAGAAGACGGCATACGAGAT,
wherein the capture primer is configured to hybridize with the capture primer.

9. The biological assay of claim 7, wherein at least one of the one or more adhesion promoting biopolymers comprises a peptide, and
wherein the peptide and capture primer form a peptide-oligo conjugate via an adaptation of the peptide and/or capture primer.

10. The biological assay of claim 9, wherein the peptide is a biomimetic adhesive peptide.

11. The biological assay of claim 10, wherein the biomimetic peptide comprise more or more of (1) polydopamine, (2) domains of poly-tyrosine with post-translational modification from tyrosine to 3,4-dihydroxyphenylalanine (also known as L-DOPA, and abbreviated as “Y*”), and (3) repeat units of Y*-X, where X is a cationic amino acid such as lysine, arginine, or histidine.

12. The biological assay of claim 9, wherein the adaptation comprises a linker attached to capture primer, wherein the linker is adapted to bind to the peptide.

13. The biological assay claim 8, wherein the biological assay is supported on a flow cell system comprising a support layer having an array of nanowells impressed in a surface of the support layer, each nanowell comprising a reaction site, wherein the functionalized solid support surface is coterminous with at least a portion of the reaction sites.

14. The biological assay of claim 13, wherein the support layer is an optic support layer configured for performing an optical detection the nucleic acid sample.

15. A method for optical detection of a nucleic acid sample, the method comprising:
providing a biological assay comprising:
a functionalized solid support surface, wherein the solid support surface comprises a hydrogel layer adhered to the surface, wherein the hydrogel contains one or more adhesion promoting biopolymers;
a capture primer covalently bound to the solid support surface through interaction with the one or one or more adhesion promoting biopolymers; and
a nucleic acid sample immobilized on the solid support surface via interaction with the capture primer, wherein the nucleic acid samples comprises one or more fluorescent species.
providing an optical detection system comprising an excitation source, one or more optical sensors, and a signal processor;
irradiating, via the excitation source, at least a portion of the functionalized solid support surface with an incident light;
detecting, via the one or more optical sensors, an output signal emitted by at least one of the one or more fluorescent species of the nucleic acid sample or constituent analyte(s) as an optic field response to the incident light; and
obtaining from the output signal, via the signal processor, data indicative of a characteristic of the sample or constituent analyte(s).

16. The method of claim 15, wherein at least one of the one or more adhesion promoting biopolymers comprises an oligonucleotide or oligonucleotide mixture, each having a nucleobase sequences of one of:
(SEQ ID No: 1)
TTTTTTTTTTAATGATACGGCGACCACCGAGAUCTACAC,
(SEQ ID No: 2)
TTTTTTAATGATACGGCGACCACCGAGA/vinyl-T/CTACAC,
and
(SEQ ID No: 3)
TTTTTTTTTTCAAGCAGAAGACGGCATACGAGAT,
wherein the capture primer is configured to hybridize with the capture primer.

17. The method of claim 15, wherein at least one of the one or more adhesion promoting biopolymers comprises a peptide, and
wherein the peptide and capture primer form a peptide-oligo conjugate via an adaptation of the peptide and/or capture primer.

18. The method of claim 17, wherein the peptide is a biomimetic adhesive peptide comprising one more or more of (1) polydopamine, (2) domains of poly-tyrosine with post-translational modification from tyrosine to 3,4-dihydroxyphenylalanine (also known as L-DOPA, and abbreviated as “Y*”), and (3) repeat units of Y*-X, where X is a cationic amino acid such as lysine, arginine, or histidine.

19. The method of claim 17, wherein the adaptation comprises a linker attached to capture primer, wherein the linker is adapted to bind to the peptide.

20. The biological assay of claim 15, wherein the biological assay is supported on a flow cell system comprising an optic support layer having an array of nanowells impressed in a surface of the support layer, each nanowell comprising a reaction site, wherein the functionalized solid support surface is coterminous with at least a portion of the reaction sites.


SEQUENCE LISTING (3 sequences)

SEQ ID NO: 1 - DNA, 39 bp, synthetic construct
  source 1..39: mol_type = other DNA
  modified_base 33: mod_base = OTHER; note = Uracil
ttttttttttaatgatacggcgaccaccgagatctacac

SEQ ID NO: 2 - DNA, 35 bp, synthetic construct
  source 1..35: mol_type = other DNA
  modified_base 29: mod_base = OTHER; note = Vinylthymine
ttttttaatgatacggcgaccaccgagatctacac

SEQ ID NO: 3 - DNA, 34 bp, synthetic construct
  source 1..34: mol_type = other DNA
ttttttttttcaagcagaagacggcatacgagat